(19) United States
(12) Patent Application Publication
(10) Pub. No.: US 2021/0343007 A1
(43) Pub. Date: Nov. 4, 2021

(54) QUALITY CONTROL METHOD AND SYSTEM FOR REMOTE FUNDUS SCREENING, AND STORAGE DEVICE

(71) Applicant: FUZHOU YIYING HEALTH TECHNOLOGY CO., LTD., Fuzhou (CN)

(72) Inventors: LUN YU, Fuzhou (CN); YING-QIANG QIU, Fuzhou (CN); JIA-WEN LIN, Fuzhou (CN); XIN-RONG CAO, Fuzhou (CN); LI-NA WANG, Fuzhou (CN)

(73) Assignee: FUZHOU YIYING HEALTH TECHNOLOGY CO., LTD., Fuzhou (CN)

(21) Appl. No.: 17/271,610

(22) PCT Filed: Nov. 28, 2018
(86) PCT No.: PCT/CN2018/117915

§ 371 Date: Feb. 26, 2021

(30) Foreign Application Priority Data
Aug. 31, 2018 (CN) 201811011974.4

(51) Int. Cl. G06T 7/00; H04N 7/18; A61B 3/12; A61B 3/14; G16H 30/20; G16H 50/20

(52) U.S. Cl. CPC G06T 7/0012; H04N 7/183; A61B 3/12; G06T 2207/30168; G16H 30/20; G16H 50/20; G06T 2207/30041; A61B 3/14

(57) ABSTRACT

A quality control method for remote fundus screening includes acquiring a fundus image of an examinee through a fundus camera by terminal application institution and transmitting the fundus image as well as personal information and inquiry data which are carried by the examinee to a remote interpretation and consultation center. The remote interpretation consultation center determines whether the fundus image, personal information, and consultation data are qualified; if the fundus image, personal information, and consultation data are unqualified, the remote interpretation consultation center terminates the interpretation. Thus clear and usable qualified fundus image is obtained during follow-up or screening of a patient and the examinee is not needed to go back and forth or repeat the examination multiple times, unnecessary expenses or time waste is saved, and the progress or screening rate of fundus screening is not affected, guaranteeing for the effective and smooth progress of remote fundus medical screening.

### BACKGROUND OF THE INVENTION

### 1. Field of the Invention

The present invention relates to the field of fundus image processing, and in particular, to a quality control method and system for remote fundus screening, and a storage device.

### 2. Description of the Prior Art

Today, the fundus camera technology used for diabetic retinopathy (DR) screening is mature. Moreover, DR screening has prevention and treatment guidelines and diagnostic standards that are related to diabetes and can guide treatment. DR screening can reduce the risk of blindness by 94.4%, but due to the lack of fundus reading or ophthalmology specialists in many primary medical institutions around the world, the DR screening rate is generally not high in many countries, and even less than 10% in China! The experience of various countries has shown: “Telemedicine is an effective way to carry out DR screening and improve the screening rate and patient compliance”! Eyes are the only part of the human body where blood vessels and nerves can be seen directly without surgery. Medical evidence shows that the circulatory system of the retina and the brain have similar anatomical, physiological and embryonic development characteristics. Therefore, through the fundus blood vessels, we can understand the disease degree of the whole body, especially the cerebral arteries and the middle and small arteries of the whole body. The changes of the fundus blood vessels are also the main basis for the risk stratification of hypertension. At the same time, the fundus camera can also be used for glaucoma, cataract (surgery maturity) and Remote screening of fundus diseases or vascular changes related to cerebral cardiovascular disease.

The application of fundus cameras to obtain fundus images is an important first step in realizing the above-mentioned remote medical fundus screening. However, the quality of health medical information such as fundus images collected or uploaded by primary medical institutions and health care services is in the process of remote screening. A common phenomenon that causes the examinee to go back and forth or repeat the examination for many times, which seriously affects the smooth progress of remote screening, causes unnecessary expenses or waste of time, and even affects the process or screening rate of fundus screening. According to research conducted by Beijing Tongren Hospital, about 40% of the remote fundus images and medical information uploaded by basic terminal application agencies have quality problems. In the process of image acquisition, due to the influence of human factors and eye factors such as shooting angle, cataracts, small pupils, etc., quality problems such as blurry and incomplete structure of the fundus image will appear. The quality of the collected or uploaded fundus images determines the service quality of the remote screening system, which is an important problem that is difficult to solve in remote fundus screening. In addition, those low-quality images that are difficult to interpret medically will directly reduce the reliability of subsequent analysis, cause interference with truly useful information, and affect the results and quality of subsequent services.

### SUMMARY OF THE INVENTION

Therefore, it is necessary to provide an image quality control method for solving the problems blurring, incomplete structure, poor quality and the like of fundus images acquired or uploaded by a basic or terminal application institution in remote fundus screening. The specific technical solutions are as follows:

a quality control method for remote fundus screening is characterized by including the following steps: acquiring a fundus image of an examinee through a fundus camera by a terminal application institution, and transmitting the fundus image as well as personal information and inquiry data which are carried by the examinee to a remote interpretation and consultation center; receiving the fundus image, the personal information and the inquiry data by the remote interpretation and consultation center, performing pre-interpretation on the fundus image, the personal information and the inquiry data and determining whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear; if the fundus image, the personal information and the inquiry data are qualified, transmitting a pre-interpretation conclusion to the terminal application institution and performing interpretation and consultation on the qualified fundus image; and if the fundus image, the personal information and the inquiry data are unqualified, terminating interpretation by the remote interpretation and consultation center.

Further, the “whether the fundus image is a fundus image required by the remote interpretation and consultation center, whether the fundus image is a left eye or a right eye and whether the structure of the fundus image is complete” further includes the steps: identifying and calibrating an optic disk and macula of the fundus image, determining whether the fundus image contains the optic disk and the macula according to the identification result, and if the fundus image contains the optic disk and the macula, determining that the fundus image is the fundus image required by the remote interpretation and consultation; according to the identification and calibration result of the optic disk and the macula of the fundus image, determining that the fundus image is the left eye or the right eye; determining whether the optic disk and the macula are in a preset interval of the fundus image according to the calibration result, and if the optic disk and the macula are in the preset interval of the fundus image, determining that the structure of the fundus image is complete.

Further, the “whether the fundus image is clear” further includes the steps: determining whether small vessels on the surface of the optic disk and a retinal nerve fiber layer of a posterior pole of the fundus image are discriminable, and if the small vessels on the surface of the optic disk and the retinal nerve fiber layer of a posterior pole of the fundus image are discriminable, determining that the definition of the fundus image is qualified.

Further, the “if the fundus image, the personal information and the inquiry data are qualified, transmitting a pre-interpretation conclusion to the terminal application institution and performing interpretation and consultation on the qualified fundus image; and if the fundus image, the personal information and the inquiry data are unqualified, terminating interpretation by the remote interpretation and consultation center” further includes the steps: receiving pre-interpretation qualification information by the terminal application institution and sending out prompt information to prompt that the examinee may leave or continue to rest nearby and wait for the consultation result of the remote interpretation and consultation center; if the fundus image, the personal information and the inquiry data are unqualified, the remote interpretation and consultation center transmits disqualification prompt information to the terminal application institution; and the terminal application institution receives the disqualification prompt information, and the fundus image of the examinee is re-acquired and re-uploaded to the remote interpretation and consultation center.

Further, before the “transmitting the fundus image as well as personal information and inquiry data which are carried by the examinee to a remote interpretation and consultation center”, the method further includes the steps: determining whether the terminal application institution is provided with specific software which may be configured to perform local pre-interpretation on the fundus image as well as the personal information and inquiry data which are carried by the examinee; if the terminal application institution is provided with the specific software which may be configured to perform local pre-interpretation on the fundus image as well as the personal information and inquiry data which are carried by the examinee, directly preforming local pre-interpretation on the fundus image, the personal information and the inquiry data by the terminal application institution; if the interpretation result is qualified, sending out corresponding prompt information to prompt that the examinee may leave or continue to rest nearby and wait for the consultation result of the remote interpretation center; if the terminal application institution is not provided with the specific software which may be configured to perform pre-interpretation on the fundus image as well as the personal information and inquiry data which are carried by the examinee and when determining that the terminal application institution is protocol service of or is closely relevant to the remote interpretation and consultation center and does not need to purchase the specific software, according to the agreements on the process or quality control system, enabling the terminal application institution to send out prompt information before receiving the pre-interpretation result returned by the remote interpretation and consultation center to prompt: the examinee should not leave the terminal application institution temporarily until the uploaded fundus image, the personal information and the inquiry data are qualified or the examinee continues to rest nearby and wait for the remote interpretation and consultation result of the remote interpretation and consultation center.

To solve the above problem, a storage device is further provided. The storage device is specifically as follows:

A storage device stores an instruction set. The instruction set is configured to perform: acquiring the fundus image, the personal information and the inquiry data of the examinee transmitted by a remote terminal; performing pre-interpretation on the fundus image, the personal information and the inquiry data and determining whether the fundus image, the personal information and the inquiry data are qualified, wherein the pre-interpretation includes one or more of the followings: whether the fundus image is a required fundus image, whether the fundus image is a left eye or a right eye, whether the structure of the fundus image is complete and whether the fundus image is clear; if the fundus image, the personal information and the inquiry data are qualified, transmitting a pre-interpretation conclusion to the remote terminal and performing interpretation and consultation on the qualified fundus image; and if the fundus image, the personal information and the inquiry data are unqualified, terminating interpretation.

Further, the instruction set is configured to further perform: the “whether the fundus image is a fundus image required by the remote interpretation and consultation center, whether the fundus image is a left eye or a right eye and whether the structure of the fundus image is complete” further includes the steps: identifying and calibrating an optic disk and macula of the fundus image, determining whether the fundus image contains the optic disk and the macula according to the identification result, and if the fundus image contains the optic disk and the macula, determining that the fundus image is the fundus image required by the remote interpretation and consultation; according to the identification and calibration result of the optic disk and the macula of the fundus image, determining that the fundus image is the left eye or the right eye; determining whether the optic disk and the macula are in a preset interval of the fundus image according to the calibration result, and if the optic disk and the macula are in the preset interval of the fundus image, determining that the structure of the fundus image is complete.

Further, the instruction set is configured to further perform: the “whether the fundus image is clear” further includes the steps: determining whether small vessels on the surface of the optic disk and a retinal nerve fiber layer of a posterior pole of the fundus image are discriminable, and if the small vessels on the surface of the optic disk and the retinal nerve fiber layer of a posterior pole of the fundus image are discriminable, determining that the definition of the fundus image is qualified.

To solve the above problem, a quality control system for remote fundus screening is provided. The specific technical solution is as follows:

Further, a quality control system for remote fundus screening includes: a fundus image acquisition terminal and a remote interpretation center terminal; the fundus image acquisition terminal is connected to the remote interpretation center terminal; the fundus image acquisition terminal includes: a fundus image acquisition module and a fundus image transmission module; the fundus image acquisition module is configured to acquire the fundus image of the examinee and transmit the fundus image to the fundus image transmission module; the fundus image transmission module is configured to transmit the fundus image as well as the personal information and inquiry data which are carried by the examinee to the remote interpretation center terminal; the remote interpretation center terminal is configured to perform pre-interpretation on the fundus image, the personal information and the inquiry data and determine whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear; if the fundus image, the personal information and the inquiry data are qualified, a pre-interpretation consultation is transmitted to the fundus image acquisition terminal and the qualified fundus image is subjected to interpretation and consultation; and if the fundus image, the personal information and the inquiry data are unqualified, interpretation is terminated.

Further, the fundus image acquisition module is a fundus camera; and the fundus image transmission module is a computer.

The present invention has the following beneficial effects: whether the fundus image is qualified and meets the requirements of the interpretation and consultation is determined by performing pre-interpretation on the fundus image of the examinee. In the remote interpretation and consultation center, the quality control system or system, an automatic or semi-automatic interactive human-computer interaction mode or image processing system may be adopted, so that the work accuracy and reliability of the quality control system or system are further improved. The key lies in the terminal, the remote interpretation and consultation center, the interaction between the terminal and the remote interpretation and consultation center, and the constitution of the quality control process or the quality control system, so as to ensure reliable pre-interpretation on the fundus image of the examinee for analysis and diagnosis or the necessary personal information and inquiry data and determine whether the fundus image or the personal information and inquiry data is qualified and meet the requirements of the remote interpretation and consultation.

The examinees with unclear images or to small pupils may take a step back to be subjected to anterior segment photography and remote screening of maturity of cataract surgery, or be subjected to fundus photography after the pupils are expanded with liquid medicine under the close attention of medical staff.

For the examinees, it is avoided that the fundus image is not available in the later stage of the diagnosis center and the examinee has to go back again, which gives very bad experience to users and waste the time of the users. For the analysis and diagnosis center, the available fundus image ensures the stability and accuracy of the diagnosis result, improves the diagnosis efficiency and avoids the useless rework time.

On the premise of computer automatic analysis on the pre-interpretation of the fundus image and in combination with the pre-interpretation information of professional quality inspection personnel, a conclusion of whether the fundus image is available is finally given. It is ensured that the finally qualified fundus images are 100% available. This double insurance on control of the quality of the fundus image is a brand-new fundus image quality control system, which has never been done by any one before.

Before the pre-interpretation result of the fundus image is returned, the examinee should wait near the fundus image acquisition terminal according to the agreement. After the pre-interpretation result is generated, whether the pre-interpretation result is qualified or unqualified, corresponding prompt information is generated and transmitted to the fundus image acquisition terminal. An operator of the terminal application institution may perform corresponding adjustment by checking the returned prompt information, or may re-acquire the fundus image and the related necessary personal information or inquiry data to ensure the smooth progress of the remote fundus medical screening. After the operator of the terminal application institution in charge of acquiring the fundus image receives the corresponding pre-interpretation result and prompt information transmitted by the remote interpretation and consultation center, the corresponding prompt information must be returned to the remote interpretation and consultation center, so that misoperation of the operator of the terminal application institution, violation of relevant quality control process or programs and unauthorized absence of the operator of the terminal application institution may be avoided.

### BRIEF DESCRIPTION OF THE DRAWINGS

FIG. 1 is a flowchart of a quality control method for remote fundus screening according to specific embodiments;

FIG. 2 is a block diagram of a storage device according to a specific embodiment;

FIG. 3 is a module connection diagram of a quality control system for remote fundus screening according to specific embodiments.

### DESCRIPTION OF REFERENCE SIGNS

200. Storage device,

300. Image quality control system;

301. Fundus image acquisition terminal;

302. Remote interpretation center terminal;

3010. Fundus image acquisition module;

3011. Fundus image transmission module.

### DETAILED DESCRIPTION OF THE INVENTION

To describe the technical contents, the structural features, the achieved objective and effect in detail, the following will perform description in detail with reference to the specific embodiments and the accompanying drawings.

The key idea of the present invention is that the fundus images uploaded by multiple quality control systems such as the remote terminal application institution are checked by the operator of the basic or terminal application institution, the remote interpretation and consultation center or the pre-interpretation personnel of the remote interpretation and consultation center, the computer aided system, the relevant quality control system and the like, thereby ensuring that the fundus images finally for analysis and determination of diseases are 100% available.

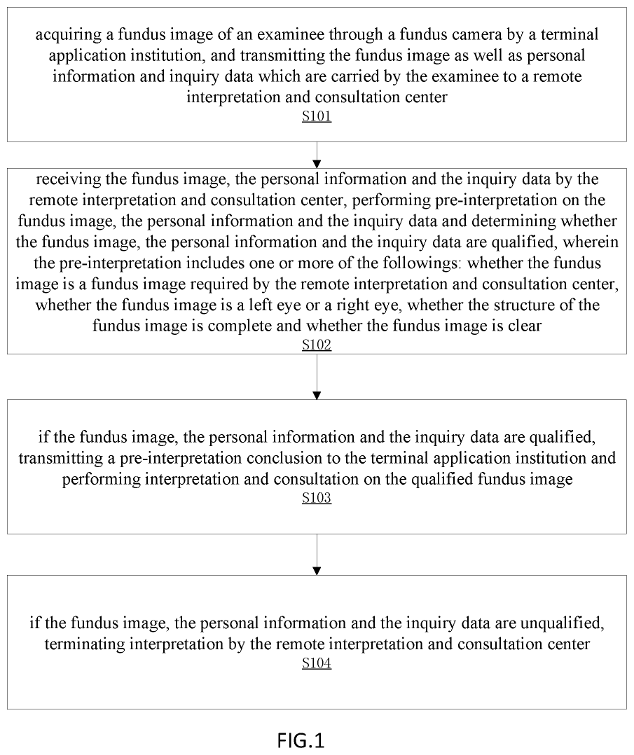

Referring to FIG. 1, in the embodiment, an application scene of a quality control method for remote fundus screening is: a basic or terminal application institution, a fundus camera and a PC capable of communicating with the fundus camera (of course, may communicate through physical connection: such as a USB interface) which are arranged in the basic or terminal application institution, and then a remote interpretation and consultation center connected to the PC computer. Specifically, for example, a health clinic in a remote area is the terminal application institution, and the remote interpretation and consultation center may be located in Beijing.

In the embodiment, a quality control method for remote fundus screening is specifically implemented as follows:

step S101: the terminal application institution acquires a fundus image of an examinee through a fundus camera and transmits the fundus image as well as personal information and inquiry data which are carried by the examinee to a remote interpretation and consultation center. The step S101 may adopt the following method: the fundus image of an examinee A is acquired by the fundus camera and is transmitted to a PC of the terminal application institution through a USB interface, and the corresponding personal information and inquiry data are input to the PC and are uniformly transmitted to the remote interpretation and consultation center by the PC.

In other embodiments, according to the interpretation and consultation content or different objectives requested by the terminal application institution, when the fundus image is transmitted to the remote interpretation and consultation center, necessary personal information or necessary other information or data may be carried.

After transmission, step S102 is performed: the remote interpretation and consultation center receives the fundus image, the personal information and the inquiry data, performs pre-interpretation on the fundus image, the personal information and the inquiry data, and determines whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear.

The step S102 may adopt the following method:

identifying and calibrating an optic disk and macula of the fundus image, determining whether the fundus image contains the optic disk and the macula according to the identification result, and if the fundus image contains the optic disk and the macula, determining that the fundus image is the fundus image required by the remote interpretation and consultation; according to the identification and calibration result of the optic disk and the macula of the fundus image, determining that the fundus image is the left eye or the right eye; determining whether the optic disk and the macula are in a preset interval of the fundus image according to the calibration result, and if the optic disk and the macula are in the preset interval of the fundus image, determining that the structure of the fundus image is complete.

The details are as follows:

The to-be-examined fundus image is subjected to green channel selection, median filtering, limited contrast enhancement and normalization processing of gray scale. By preprocessing the fundus image, the redundant background in the fundus image may be removed and the noise may be effectively removed, which is more beneficial to the subsequent fundus image analysis. The details are as follows: In any color fundus image, under a blue channel, there is more noise and useful information is basically lost; and under a red channel, two spots are more prominent and information such as dark hemangioma, hemangioma capillanisum and the like are more lost. Therefore, in the embodiment, a green channel is selected for the to-be-examined color fundus image, thereby retaining and highlighting the fundus blood vessels maximally.

To remove the noise and retain the boundary information well, in this embodiment, the fundus image under the green channel is subjected to median filtering to remove noise.

To achieve better blood vessel extraction effect, the denoised image is subjected to contrast enhancement. In this embodiment, a limited contrast enhancement method CLAHE (Contrast Limited Adaptive Histogram Equalization) is used to avoid over-brightness after image enhancement. Finally, normalization processing is performed, so that pixel values of all pixel points in one image fall between 0 and 1.

A binary vascular image is extracted from the preprocessed fundus image by an otsu algorithm and the binary vascular image is corroded by a morphological method to obtain a main blood vessel, which may adopt the following method: a threshold of the preprocessed fundus image is calculated by the otsu method, and a pixel with a grayscale greater than the threshold is identified as a blood vessel according to the following formula; and

$${Map}_{v} ( i , j ) = \{ \begin{matrix}1 , & if Gv ( i , j ) > T \\ 0 , & otherwise\end{matrix}$$

a structural element is constructed according to a diameter of the optic disk being ⅛ to ⅕ of a width of the image and a width of the main blood vessel being ¼ of the diameter of the optic disk, and the extracted blood vessel is subjected to corrosion operation by the structural element to remove fine blood vessels to obtain the main blood vessel.

After the main blood vessel is obtained, the main blood vessel is subjected to parabolic fitting calculation, and a center of the optic disk is positioned according to the calculation result, which may adopt the following method: a coordinate system is established with the upper left corner of the fundus image as an original point, a horizontal direction as an X axis and a vertical direction as a Y axis;

each pixel point in the main blood vessel is mapped as coordinates of the coordinate system;

as shown in the following formula, the main blood vessel is subjected to parabolic fitting according to a least square method, parameters of the parabola are determined, and a vertex of the parabola is calculated; and

f(x)=ax²+bx+c

S(a,b,c)=Σᵢ₌₁^{N}[f(xᵢ)−yᵢ]²

whether the vertex of the parabola falls in the original fundus image is determined, if the vertex of the parabola falls in the original fundus image, the vertex of the parabola is defined as the center of the optic disk.

The macular positioning based on appearance and structure features: according to the position relationship between the macula and the optic disk, firstly, the search range of the fovea is further shortened based on the determined center of the optic disk. In a preferred manner, since a distance between the macular fovea and the center of the optic disk is generally 2 to 3 times a diameter of the optic disk, an annular mask is constructed by taking the center of the optic disk as a circle center and is defined as the search range of the fovea; and then in the search range area, the fovea is positioned according to the characteristic that the fovea has the lowest brightness. In a preferred manner, the position of the fovea is determined by a rapid search method based on brightness contrast between areas; and finally, a macular area is fitted circularly with the fovea as a circle center according to the brightness information.

After the macula and the center of the optic disk are determined, the integrity of the structure of the fundus image is determined. According to the determination condition shown in Table 1, the image meeting the condition is an image with qualified integrity. Dod is the diameter of the optic disk.

TABLE 1

| Different | | Determination |
|---|---|---|
| Distance | Description | Condition |
| D_{OD-FOVEA} | Distance from the | 1.5D_{OD} < |
| | center of the | D_{OD-FOVEA} < |
| | optic disk to the | 3.5D_{OD} |
| | center of the | |
| | macula | |
| D_{OD-EDGE} | Distance from the | 1D_{OD} < |
| | center of the | D_{OD-EDGE} |
| | optic disk to the | |
| | edge | |
| D_{FOVEA-EDGE} | Distance from the | 2.5D_{OD} < |
| | center of the | D_{FOVEA-EDGE} |
| | macula to the | |
| | edge | |

If the fundus image is the fundus image, whether the structure of the fundus image is clear is determined, which may adopt the following method: whether small vessels on the surface of the optic disk and a retinal nerve fiber layer of a posterior pole of the fundus image are discriminable is determined, and if the small vessels on the surface of the optic disk and the retinal nerve fiber layer of a posterior pole of the fundus image are discriminable, the definition of the fundus image is determined to be qualified. The details are as follows:

a. according to the identified optic disk center and macular fovea, an area with the optic disk as a circle center and 1.5 times the diameter of the optic disk is determined as an interested area, and an area with the macular fovea as a circle center and 1 time the diameter of the optic disk is determined as an interested area 2; and

b. based on the determined interested area 1 and the interested area 2, a certain definition evaluation operator is selected, a definition evaluation value is calculated and definition evaluation is completed.

Step S103: if the fundus image, the personal information and the inquiry data are qualified, a pre-interpretation conclusion is transmitted to the terminal application mechanism and the qualified fundus image is subjected to pre-interpretation and consultation.

Step S104: if the fundus image, the personal information and the inquiry data are unqualified, the remote interpretation and consultation center terminates interpretation.

The details are as follows:

the terminal application institution receives the pre-interpretation qualification information and sends out prompt information to prompt that the examinee may leave or continue to rest nearby and wait for the consultation result of the remote interpretation and consultation center;

if the fundus image, the personal information and the inquiry data are unqualified, the remote interpretation and consultation center transmits disqualification prompt information to the terminal application institution; and

the terminal application institution receives the disqualification prompt information, and the fundus image of the examinee is re-acquired and re-uploaded to the remote interpretation and consultation center.

Before the “transmitting the fundus image as well as personal information and inquiry data which are carried by the examinee to a remote interpretation and consultation center”, the method further includes the steps:

whether the terminal application institution is provided with specific software which may be configured to perform local pre-interpretation on the fundus image as well as the personal information and inquiry data which are carried by the examinee is determined; if the terminal application institution is provided with the specific software which may be configured to perform local pre-interpretation on the fundus image as well as the personal information and inquiry data which are carried by the examinee, the terminal application institution directly preforms local pre-interpretation on the fundus image, the personal information and the inquiry data by the terminal application institution; if the interpretation result is qualified, corresponding prompt information is sent out to prompt that the examinee may leave or continue to rest nearby and wait for the consultation result of the remote interpretation center; and

if the terminal application institution is not provided with the specific software which may be configured to perform pre-interpretation on the fundus image as well as the personal information and inquiry data which are carried by the examinee and when it is determined that the terminal application institution is protocol service of or is closely relevant to the remote interpretation and consultation center and does not need to purchase the specific software, according to the agreements on the process or quality control system, the terminal application institution sends out prompt information before receiving the pre-interpretation result returned by the remote interpretation and consultation center to prompt: the examinee should not leave the terminal application institution temporarily until the uploaded fundus image, the personal information and the inquiry data are qualified or the examinee continues to rest nearby and wait for the remote interpretation and consultation result of the remote interpretation and consultation center.

The following method may be adopted:

if the terminal application institution receives that the pre-interpretation conclusion fed back by the remote interpretation and consultation center is unqualified, it is necessary to consider whether to re-acquire and re-upload the fundus image as well as accompanied other personal information and inquiry data; and

after the terminal application institution acquires the fundus image and the accompanied other personal information or before the terminal application institution uploads the fundus image and the accompanied other personal information, local automatic pre-interpretation may be performed by online or random “local quality control automatic interpretation program”, if the result is qualified, the examinee may leave or continue to rest nearby and wait for the remote interpretation and consultation result of the remote interpretation and consultation center, wherein the automatic interpretation function of the “local quality control automatic interpretation program” is as same as the function of quality control automatic interpretation program software of the remote interpretation and consultation center.

If the terminal application institution does not purchase or have the online or random “local quality control automatic interpretation program” software and when the terminal application institution is protocol service of or is closely relevant to the remote interpretation and consultation center and does not need to purchase the specific software, the agreement of the process or quality control system may be utilized, when the terminal application institution uploads the fundus image and accompanied other personal information and inquiry data and waits for the pre-interpretation result of the remote interpretation and consultation center, the examinee does not leave the terminal application institution temporarily until the pre-interpretation of the acquired fundus image and the uploaded data is qualified, or the examinee continues to rest nearby and wait for the remote interpretation and consultation result of the remote interpretation and consultation center; and

After the terminal application institution uploads the fundus image and accompanied other personal information and inquiry data and receives that the pre-interpretation conclusion fed back by the center is qualified, the examinee may leave or continue to rest nearby and wait for the remote interpretation and consultation result of the remote interpretation and consultation center.

Whether the fundus image is qualified and meets the requirements of the interpretation and consultation is determined by performing pre-interpretation on the fundus image of the examinee. In the remote interpretation and consultation center, the quality control system or system, an automatic or semi-automatic interactive human-computer interaction mode or image processing system may be adopted, so that the work accuracy and reliability of the quality control system or system are further improved. The key lies in the terminal, the remote interpretation and consultation center, the interaction between the terminal and the remote interpretation and consultation center, and the constitution of the quality control process or the quality control system, so as to ensure reliable pre-interpretation on the fundus image of the examinee for analysis and diagnosis or the necessary personal information and inquiry data and determine whether the fundus image or the personal information and inquiry data is qualified and meet the requirements of the remote interpretation and consultation.

The examinees with unclear images or to small pupils may take a step back to be subjected to anterior segment photography and remote screening of maturity of cataract surgery, or be subjected to fundus photography after the pupils are expanded with liquid medicine under the close attention of medical staff.

For the examinees, it is avoided that the fundus image is not available in the later stage of the diagnosis center and the examinee has to go back again, which gives very bad experience to users and waste the time of the users. For the analysis and diagnosis center, the available fundus image ensures the stability and accuracy of the diagnosis result, improves the diagnosis efficiency and avoids the useless rework time.

On the premise of computer automatic analysis on the pre-interpretation of the fundus image and in combination with the pre-interpretation information of professional quality inspection personnel, a conclusion of whether the fundus image is available is finally given. It is ensured that the finally qualified fundus images are 100% available. This double insurance on control of the quality of the fundus image is a brand-new fundus image quality control system, which has never been done by any one before.

Before the pre-interpretation result of the fundus image is returned, the examinee should wait near the fundus image acquisition terminal according to the agreement. After the pre-interpretation result is generated, whether the pre-interpretation result is qualified or unqualified, corresponding prompt information is generated and transmitted to the fundus image acquisition terminal. An operator of the terminal application institution may perform corresponding adjustment by checking the returned prompt information, or may re-acquire the fundus image and the related necessary personal information or inquiry data to ensure the smooth progress of the remote fundus medical screening. After the operator of the terminal application institution in charge of acquiring the fundus image receives the corresponding pre-interpretation result and prompt information transmitted by the remote interpretation and consultation center, the corresponding prompt information must be returned to the remote interpretation and consultation center, so that misoperation of the operator of the terminal application institution, violation of relevant quality control process or programs and unauthorized absence of the operator of the terminal application institution may be avoided.

Pre-interpretation of the fundus image may be performed by an automatic or semi-automatic interactive human-computer interaction mode or system, so that the work accuracy and reliability of the quality control system or system are further improved. On the premise of the assistance of the computer and in combination with pre-interpretation information of professional quality inspection personnel, a conclusion of whether the fundus image is available is finally given. It is ensured that the finally qualified fundus images are 100% available. This double insurance of the quality control system process is a brand-new fundus image quality control system, which has never been done by any one before.

In other embodiments, one process of the fundus image quality control method for remote fundus screening meets and includes the following steps (wherein necessary personal information or inquiry data has been nondestructively hidden in the uploaded fundus image by a privacy protection method, the remote interpretation and consultation center receives and decode the personal information or inquiry data and pre-determines the integrity or availability, and these contents are not described in the following steps):

a. the basic terminal application institution transmits the acquired fundus image to the center and asks the examinee to wait aside; the remote interpretation and consultation center performs pre-interpretation and records an image source after receiving the fundus image transmitted by the basic terminal;
b. for each of the received fundus images, the center may set or not set the link of “firstly determining whether the fundus image is the fundus image”, or may also cross this link with a step d; if the link is set and the image quality is determined not to the fundus image, (it may be mischief of the personnel of the basic terminal application institution or misoperation when the fundus image is selected for transmission) this interpretation and consultation ends or enters the step c; and if the determination result is the fundus image, the step d is performed and the interpretation and consultation will continue; and
c. the remote interpretation and consultation center interprets that the consultation center system feeds back the disqualification consultation or specific reasons (for example, the non-fundus image is transmitted or warning is performed, the structure is incomplete, and the image is unclear or does not have availability) to the image source position (basic terminal) recorded in the step a, requires to re-select a correct fundus image which should be transmitted or requires the basic terminal to re-acquire the unqualified fundus image until the fundus image and the relevant inquiry data transmitted to the remote interpretation and consultation center are qualified or the fundus image of the examinee cannot be continuously acquired, the examinee leaves or continues to remains and wait for the interpretation and consultation result sent back by the remote interpretation and consultation center.
d. The center staff combines with the automatic analysis or human-computer interaction interpretation result of the computer and finally gives the evaluation conclusion of the image quality and the availability. If qualified, the step e is performed; and if not qualified, the step c is performed.
e. The optic disk or the macula is identified or calibrated. According to the stipulated standard of the quality of single or relevant fundus image in the remote fundus image screening system, whether the structure or integrity of the fundus image meets the requirement is determined. If the determination result does not meet the requirement, the step c is performed; and if the determination result meets the requirement, the step f is performed.
f. The definition of the fundus image is analyzed (specifically, the fundus photography may be required to focus accurately, the main retinal vessel and the retinal nerve fiber layer on the fundus image of normal people are clear, and that small vessels on the surface of the optic disk and a retinal nerve fiber layer of a posterior pole of the fundus image are discriminable is taken as reference), and the definition of the fundus image is determined according to the stipulated standard of the quality of single or relevant fundus images in the remote fundus image screening system. If the definition does not meet the requirement, the step c is performed; if the definition meets the requirement, the step g is performed; the order of the step process may be exchanged with the step e, but in general, completing the step e before this step is beneficial to the selection the interested areas in the computer automatic analysis of the definition.
g. According to the stipulated standard of the quality of single or relevant fundus image in the remote fundus image screening system, after the comprehensive determination result of the structure, integrity or definition analysis of the fundus image is qualified, or the comprehensive determination result of the structure, integrity or definition analysis of the transmitted inquiry data and the fundus image is qualified, the conclusion is fed back to the basic health and medical institution or terminal (at this time, the basic terminal staff may ask the examinee to leave or end the screening and wait for the specific interpretation report), the center may enter the subsequent image analysis, interpretation or consultation stage; and

the fundus image quality control method or process in remote fundus medical screening may serve as a necessary link in the remote fundus medical screening system, and may also be applied to operation of the fundus camera of the basic terminal application institution and interpretation and consultation of work of medical or health service staff and be a necessary method or process to realize image quality control. The application specifically includes, but is not limited to, an auxiliary determination method or process of a health big data service system, a third party image detection institution, a remote fundus interpretation and consultation platform, a cloud health service system or a fundus camera terminal based on remote fundus image examination or screening.

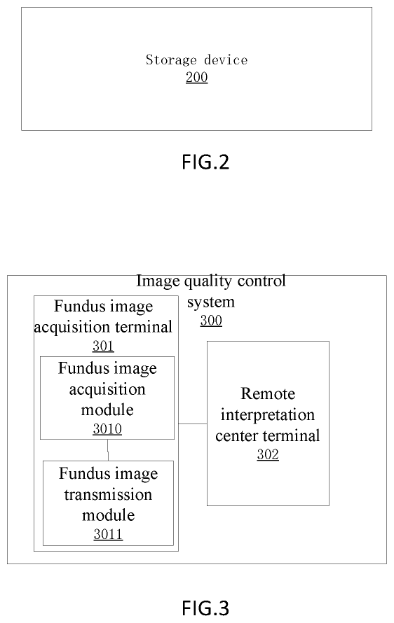

Referring to FIG. 2, a storage device 200 is specifically implemented as follows:

in this embodiment, the storage device corresponds to the steps performed by the remote interpretation center, so the details of the following steps are not elaborated.

A storage device 200 stores an instruction set. The instruction set is configured to perform: the fundus image, the personal information and the inquiry data of the examinee transmitted by a remote terminal are required; the fundus image, the personal information and the inquiry data are subjected to pre-interpretation and whether the fundus image, the personal information and the inquiry data are qualified is determined, wherein the pre-interpretation includes one or more of the followings: whether the fundus image is a required fundus image, whether the fundus image is a left eye or a right eye, whether the structure of the fundus image is complete and whether the fundus image is clear; if the fundus image, the personal information and the inquiry data are qualified, a pre-interpretation conclusion is transmitted to the remote terminal and performing interpretation and consultation on the qualified fundus image; and if the fundus image, the personal information and the inquiry data are unqualified, interpretation is terminated.

The instruction set is configured to further perform: the “whether the fundus image is a required fundus image, whether the fundus image is a left eye or a right eye and whether the structure of the fundus image is complete” further includes the steps: an optic disk and macula of the fundus image are identified and calibrated, whether the fundus image contains the optic disk and the macula is determined according to the identification result, and if the fundus image contains the optic disk and the macula, the fundus image is determined to be the fundus image required by the remote interpretation and consultation; according to the identification and calibration result of the optic disk and the macula of the fundus image, the fundus image is determined to be the left eye or the right eye; whether the optic disk and the macula are in a preset interval of the fundus image is determined according to the calibration result, and if the optic disk and the macula are in the preset interval of the fundus image, the structure of the fundus image is determined to be complete.

The instruction set is configured to further perform: the “whether the fundus image is clear” further includes the steps: determining whether small vessels on the surface of the optic disk and a retinal nerve fiber layer of a posterior pole of the fundus image are discriminable, and if the small vessels on the surface of the optic disk and the retinal nerve fiber layer of a posterior pole of the fundus image are discriminable, determining that the definition of the fundus image is qualified.

Referring to FIG. 3, a quality control system 300 for remote fundus screening includes: a fundus image acquisition terminal 301 and a remote interpretation center terminal 302; the fundus image acquisition terminal 301 is connected to the remote interpretation center terminal 302; the fundus image acquisition terminal 301 includes: a fundus image acquisition module 3010 and a fundus image transmission module 3011; the fundus image acquisition module 3010 is configured to acquire the fundus image of the examinee and transmit the fundus image to the fundus image transmission module 3011; the fundus image transmission module 3011 is configured to transmit the fundus image as well as the personal information and inquiry data which are carried by the examinee to the remote interpretation center terminal 302; and

the remote interpretation center terminal 302 is configured to perform pre-interpretation on the fundus image, the personal information and the inquiry data and determine whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear; if the fundus image, the personal information and the inquiry data are qualified, a pre-interpretation consultation is transmitted to the fundus image acquisition terminal and the qualified fundus image is subjected to interpretation and consultation; and if the fundus image, the personal information and the inquiry data are unqualified, interpretation is terminated.

The fundus image acquisition module 3010 is a fundus camera; and the fundus image transmission module 3011 is a computer.

In the embodiment, the specific implementation manner of the quality control system 300 for remote fundus screening is as same as the above quality control method for remote fundus screening, and will not be repeated here.

It should be noted that although the foregoing embodiments have been described herein, the scope of patent protection of the present invention is not limited thereby. Therefore, based on the innovative concept of the present invention, changes and modifications to the embodiments described herein, or equivalent structures or equivalent process transformations made by using the description and drawings of the present invention, directly or indirectly apply the above technical solutions In other related technical fields, they are all included in the scope of patent protection of the present invention.

What is claimed is:

1. A quality control method for remote fundus screening is characterized by including the following steps:
acquiring a fundus image of an examinee through a fundus camera by a terminal application institution, and transmitting the fundus image as well as personal information and inquiry data which are carried by the examinee to a remote interpretation and consultation center;
receiving the fundus image, the personal information and the inquiry data by the remote interpretation and consultation center, performing pre-interpretation on the fundus image, the personal information and the inquiry data and determining whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear;
if the fundus image, the personal information and the inquiry data are qualified, transmitting a pre-interpretation conclusion to the terminal application institution and performing interpretation and consultation on the qualified fundus image;
and if the fundus image, the personal information and the inquiry data are unqualified, terminating interpretation by the remote interpretation and consultation center.

2. A quality control method for remote fundus screening according to claim 1, wherein the “whether the fundus image is a fundus image required by the remote interpretation and consultation center, whether the fundus image is a left eye or a right eye and whether the structure of the fundus image is complete” further includes the steps:
identifying and calibrating an optic disk and macula of the fundus image, determining whether the fundus image contains the optic disk and the macula according to the identification result, and if the fundus image contains the optic disk and the macula, determining that the fundus image is the fundus image required by the remote interpretation and consultation;
determining that the fundus image is the left eye or the right eye according to the identification and calibration result of the optic disk and the macula of the fundus image;
determining whether the optic disk and the macula are in a preset interval of the fundus image according to the calibration result, and if the optic disk and the macula are in the preset interval of the fundus image, determining that the structure of the fundus image is complete.

3. A quality control method for remote fundus screening according to claim 1, wherein the “whether the fundus image is clear” further includes the steps:
determining whether small vessels on the surface of the optic disk and a retinal nerve fiber layer of a posterior pole of the fundus image are discriminable, and if the small vessels on the surface of the optic disk and the retinal nerve fiber layer of a posterior pole of the fundus image are discriminable, determining that the definition of the fundus image is qualified.

4. A quality control method for remote fundus screening according to claim 1, wherein the “if the fundus image, the personal information and the inquiry data are qualified, transmitting a pre-interpretation conclusion to the terminal application institution and performing interpretation and consultation on the qualified fundus image; and if the fundus image, the personal information and the inquiry data are unqualified, terminating interpretation by the remote interpretation and consultation center” further includes the steps:
receiving pre-interpretation qualification information by the terminal application institution and sending out prompt information to prompt that the examinee may leave or continue to rest nearby and wait for the consultation result of the remote interpretation and consultation center;
transmitting disqualification prompt information to the terminal application institution by the remote interpretation and consultation center if the fundus image, the personal information and the inquiry data are unqualified; and
re-acquiring and re-uploading the fundus image of the examinee, by the terminal application institution, to the remote interpretation and consultation center after receiving the disqualification prompt information.

5. A quality control method for remote fundus screening according to claim 1, wherein before the “transmitting the fundus image as well as personal information and inquiry data which are carried by the examinee to a remote interpretation and consultation center”, the method further includes the steps:
determining whether the terminal application institution is provided with specific software which may be configured to perform local pre-interpretation on the fundus image as well as the personal information and inquiry data which are carried by the examinee;
if the terminal application institution is provided with the specific software which may be configured to perform local pre-interpretation on the fundus image as well as the personal information and inquiry data which are carried by the examinee, directly preforming local pre-interpretation on the fundus image, the personal information and the inquiry data by the terminal application institution;
if the interpretation result is qualified, sending out corresponding prompt information to prompt that the examinee may leave or continue to rest nearby and wait for the consultation result of the remote interpretation center;
if the terminal application institution is not provided with the specific software which may be configured to perform pre-interpretation on the fundus image as well as the personal information and inquiry data which are carried by the examinee and when determining that the terminal application institution is protocol service of or is closely relevant to the remote interpretation and consultation center and does not need to purchase the specific software, according to the agreements on the process or quality control system, enabling the terminal application institution to send out prompt information before receiving the pre-interpretation result returned by the remote interpretation and consultation center to prompt: the examinee should not leave the terminal application institution temporarily until the uploaded fundus image, the personal information and the inquiry data are qualified or the examinee continues to rest nearby and wait for the remote interpretation and consultation result of the remote interpretation and consultation center.

6. A storage device stores an instruction set, wherein the instruction set is configured to perform:
acquiring the fundus image, the personal information and the inquiry data of the examinee transmitted by a remote terminal;
performing pre-interpretation on the fundus image, the personal information and the inquiry data and determining whether the fundus image, the personal information and the inquiry data are qualified, wherein the pre-interpretation includes one or more of the followings: whether the fundus image is a required fundus image, whether the fundus image is a left eye or a right eye, whether the structure of the fundus image is complete and whether the fundus image is clear;
if the fundus image, the personal information and the inquiry data are qualified, transmitting a pre-interpretation conclusion to the remote terminal and performing interpretation and consultation on the qualified fundus image; and
if the fundus image, the personal information and the inquiry data are unqualified, terminating interpretation.

7. A storage device according to claim 6, wherein the “whether the fundus image is a fundus image required by the remote interpretation and consultation center, whether the fundus image is a left eye or a right eye and whether the structure of the fundus image is complete” further includes the steps:
identifying and calibrating an optic disk and macula of the fundus image, determining whether the fundus image contains the optic disk and the macula according to the identification result, and if the fundus image contains the optic disk and the macula, determining that the fundus image is the fundus image required by the remote interpretation and consultation;
determining that the fundus image is the left eye or the right eye according to the identification and calibration result of the optic disk and the macula of the fundus image;
determining whether the optic disk and the macula are in a preset interval of the fundus image according to the calibration result, and if the optic disk and the macula are in the preset interval of the fundus image, determining that the structure of the fundus image is complete.

8. A storage device according to claim 6, wherein the “whether the fundus image is clear” further includes the steps:
determining whether small vessels on the surface of the optic disk and a retinal nerve fiber layer of a posterior pole of the fundus image are discriminable, and if the small vessels on the surface of the optic disk and the retinal nerve fiber layer of a posterior pole of the fundus image are discriminable, determining that the definition of the fundus image is qualified.

9. A quality control system for remote fundus screening includes: a fundus image acquisition terminal and a remote interpretation center terminal; the fundus image acquisition terminal is connected to the remote interpretation center terminal; the fundus image acquisition terminal includes: a fundus image acquisition module and a fundus image transmission module; the fundus image acquisition module is configured to acquire the fundus image of the examinee and transmit the fundus image to the fundus image transmission module; the fundus image transmission module is configured to transmit the fundus image as well as the personal information and inquiry data which are carried by the examinee to the remote interpretation center terminal; the remote interpretation center terminal is configured to perform pre-interpretation on the fundus image, the personal information and the inquiry data and determine whether the fundus image, the personal information and the inquiry data are qualified, wherein the pre-interpretation includes one or more of the followings: whether the fundus image is a fundus image required by the remote interpretation and consultation center, whether the fundus image is a left eye or a right eye, whether the structure of the fundus image is complete and whether the fundus image is clear; if the fundus image, the personal information and the inquiry data are qualified, a pre-interpretation consultation is transmitted to the fundus image acquisition terminal and the qualified fundus image is subjected to interpretation and consultation; and if the fundus image, the personal information and the inquiry data are unqualified, interpretation is terminated.

10. A quality control system for remote fundus screening according to claim 9, wherein the fundus image acquisition module is a fundus camera; and the fundus image transmission module is a computer.
